(19) United States
(12) Patent Application Publication
(10) Pub. No.: US 2022/0192573 A1
(43) Pub. Date: Jun. 23, 2022

(54) ELECTROCARDIOGRAM MONITORING DEVICE AND ELECTROCARDIOGRAM MONITORING SYSTEM

(71) Applicant: GOERTEK INC., Weifang, Shandong (CN)

(72) Inventors: Xin YE, Weifang, Shandong (CN); Yujuan YANG, Weifang, Shandong (CN)

(21) Appl. No.: 17/599,807

(22) PCT Filed: Jul. 25, 2019
(86) PCT No.: PCT/CN2019/097637

§ 371 Date: Sep. 29, 2021

(30) Foreign Application Priority Data
Apr. 3, 2019 (CN) 201910269931.4

(51) Int. Cl. A61B 5/332; A61B 5/339; A61B 5/00

(52) U.S. Cl. CPC A61B 5/332; A61B 5/339; A61B 2562/221; A61B 5/6823; A61B 5/6831; A61B 5/681

(57) ABSTRACT

Disclosed are an electrocardiogram monitoring device (100) and an electrocardiogram monitoring system. The electrocardiogram monitoring device (100) comprises: a main body (10) and a bottom support, wherein the main body (10) is worn on a wrist, the main body (10) is provided with a controller and a first monitor (11) electrically connected to the controller, and the first monitor (11) is used for generating electrocardiosignals when abutting against two hands and sending the electrocardiosignals to the controller; and the bottom support is provided with a second monitor, the second monitor is in signal connection with the controller, and the second monitor is used for generating electrocardiosignals when being attached to the chest or a limb and sending the electrocardiosignals to the controller. The electrocardiogram monitoring device (100) may realize diverse detection and is easy to operate.

The present application claims priority to Chinese Patent Application No. 201910269931.4, titled “ELECTROCARDIOGRAM MONITORING DEVICE AND ELECTROCARDIOGRAM MONITORING SYSTEM”, filed on Apr. 3, 2019 with the China National Intellectual Property Agency, which is incorporated herein by reference in its entirety.

### FIELD

The present disclosure relates to the technical field of mobile medical treatment, and in particular to an electrocardiogram monitoring device and an electrocardiogram monitoring system.

### BACKGROUND

Increasing attention is paid to health under improvement of living standards. Nowadays the death toll of heart diseases increases rapidly every year. For precaution against a sudden heart attack, more and more people choose an electrocardiogram monitoring device, which is portable, user-friendly, and convenient, for out-of-hospital monitoring. Most traditional electrocardiogram monitors are worn at the waist, such as HOTER instrument, hand-held in both hands, or worn on the chest. Such devices are too bulky for daily operation of users. Some electrocardiogram monitors are worn at the wrist, and a single monitor only supports discontinuous monitoring.

### SUMMARY

An electrocardiogram monitoring device is provided according to embodiments of the present disclosure, in order to implement diverse monitoring of the electrocardiogram monitoring device and facilitate operations on the electrocardiogram monitoring device.

In view of the above, an electrocardiogram monitoring device is provided according to embodiments of the present disclosure, including:
a main body, configured to be worn on a wrist, where the main body is provided with a controller and a first monitor electrically coupled to the controller, the first monitor is configured to generate an electrocardiogram signal and transmit the electrocardiogram signal to the controller when being in contact with two hands; and
a bottom support, provided with a second monitor, where the second monitor is coupled to the controller in signals, and is configured to generate an electrocardiogram signal and transmit the electrocardiogram signal to the controller when being attached to a chest or a limb.

In an embodiment, the main body includes a case body. The first monitor includes an inside electrode, an outside electrode and a connection contact. The inside electrode is disposed on a surface, of the case body, attached to the wrist. The outside electrode is disposed on a surface, of the case body, away from the wrist. The connection contact is embedded inside the case body. The outside electrode is electrically connected to the inside electrode via the connection contact.

In an embodiment, the case body is configured to magnetically connect to the bottom support.

In an embodiment, the case body is provided with at least one first magnet, and the bottom support is provided with a second magnet corresponding to the first magnet.

In an embodiment, the bottom support includes a flexible bottom support. The main body is configured to be attached to a surface of the flexible bottom support. The second monitor is disposed on a surface of the flexible bottom support away from the main body.

In an embodiment, the bottom support includes a rigid bottom support. The second monitor includes an electrode for a lead. The electrode for the lead is electrically connected to the rigid bottom support, and is configured to be attached to the chest or the limb.

In an embodiment, the case body further includes a sensor. The flexible bottom support is provided with a first induction magnet configured to magnetically attract the case body. The rigid bottom support is provided with a second induction magnet configured to magnetically attract the case body. The sensor is configured to detect whether magnetic attraction around the case body is between the case body and the first induction magnet, or between the case body and the second induction magnet.

In an embodiment, the second monitor includes two fixed electrodes. The two fixed electrodes are disposed at two ends, respectively, of the flexible bottom support. The flexible bottom support is further provided with a flexible expansion contact electrically connected to the fixed electrodes. The flexible expansion contact is disposed in correspondence to the connection contact, and is configured to connect the connection contact electrically and elastically.

In an embodiment, the rigid bottom support is provided with a rigid expansion contact and a USB interface that are electrically connected with each other. The rigid expansion contact is disposed in correspondence to the connection contact, and is configured to connect the connection contact electrically and elastically. The electrode for the lead is electrically connected to the USB interface via a connection wire.

An electrocardiogram monitoring system is further provided according to embodiments of the present disclosure. The electrocardiogram monitoring system includes the foregoing electrocardiogram monitoring device and a display terminal coupled to the electrocardiogram monitoring device.

The electrocardiogram monitoring device according to technical solutions of the present disclosure includes the main body and the bottom support, and is capable of different monitoring modes. The main body includes the controller and the first monitor. In daytime usage, the electrocardiogram monitoring device may operate in a watch mode. The main body may be worn on the wrist corresponding to a hand as an independent gadget, while a finger of the other hand touches the first monitor of the main body to perform electrocardiogram monitoring. The electrocardiogram signal generated by the first monitor is transmitted to the controller for processing. The monitoring is stopped when inconvenient. The main body is worn on the wrist, thereby causing little inconvenience, improving user experience, and facilitating simple discontinuous electrocardiogram monitoring. In night usage or daytime rests, continuous electrocardiogram monitoring may be implemented through coordination of both hands without additional user operations. Alternatively, the main body may be electrically connected to the bottom support, so that the second monitor on the bottom support is coupled to the controller in signals. After the second monitor is attached to the chest or the limb, an electrocardiogram signal is generated and transmitted to the controller to implement continuous electrocardiogram monitoring, which is called a “chest paste” mode or an electrode mode, without operation of two hands. Hence, the electrocardiogram monitoring device can implement both continuous and discontinuous electrocardiogram monitoring, and is advantageous in flexibility, various selections, simplicity, and convenience.

### BRIEF DESCRIPTION OF THE DRAWINGS

For clearer illustration of the technical solutions according to embodiments of the present disclosure or conventional techniques, hereinafter briefly described are the drawings to be applied in embodiments of the present disclosure or conventional techniques. Apparently, the drawings in the following descriptions are only some embodiments of the present disclosure, and other drawings may be obtained by those skilled in the art based on the provided drawings without creative efforts.

FIG. 1 is a schematic diagram of an electrocardiogram monitoring device according to an embodiment of the present disclosure;

FIG. 2 is a structural schematic diagram of a main body of an electrocardiogram monitoring device as shown in FIG. 1;

FIG. 3 is a structural schematic diagram of a main body as shown in FIG. 2 in another view;

FIG. 4 is a structural schematic diagram of a bottom support of an electrocardiogram monitoring device as shown in FIG. 1;

FIG. 5 is a schematic diagram of an electrocardiogram monitoring device, as shown in FIG. 1, performing continuous electrocardiogram monitoring;

FIG. 6 is a schematic diagram of an electrocardiogram monitoring device according to another embodiment of the present disclosure; and

FIG. 7 is a structural schematic diagram of a bottom support of an electrocardiogram monitoring device as shown in FIG. 6.

### ILLUSTRATION OF REFERENCE NUMERALS

| Reference | | Reference | |
|---|---|---|---|
| numeral | Component | numeral | Component |
| 100 | Electrocardiogram | 33 | First induction |
| | monitoring device | | magnet |
| 10 | Main body | 35 | Flexible expansion |
| | | | contact |
| 11 | First monitor | 37 | Fixed electrode |
| 111 | Inside electrode | 39 | Flexible substrate |
| 113 | Outside electrode | 50 | Rigid bottom support |
| 115 | Connection contact | 51 | Electrode for lead |
| 13 | Case body | 53 | Second induction |
| | | | magnet |
| 15 | Watch strap | 55 | Rigid expansion |
| | | | contact |
| 30 | Flexible bottom | 57 | USB interface |
| | support | | |
| 31 | Second magnet | 59 | Rigid substrate |

### DETAILED DESCRIPTION OF EMBODIMENTS

Hereinafter technical solutions in embodiments of the present disclosure are described clearly and completely in conjunction with the drawings in embodiments of the present closure. Apparently, the described embodiments are only some rather than all of the embodiments of the present disclosure. Any other embodiments obtained based on the embodiments of the present disclosure by those skilled in the art without any creative effort fall within the scope of protection of the present disclosure.

It should be noted that all directional terms (such as top, bottom, left, right, front, back, etc.) in embodiments of the present disclosure are only intended to illustrate a relative position relationship, relative motion, or the like between different components under a particular attitude (as shown in the drawings). The directional terms may change in case of a different attitude.

In the present disclosure, unless clearly defined or described otherwise, the terms “connection” and “fixation” should be broadly interpreted. For example, unless clearly defined otherwise, “fixation” may be a fixed connection, a detachable connection, or connection as an integral, may be a mechanical connection or an electrical connection, may be a direct connection, an indirect connection via a medium, or an internal connection or interaction between two elements. Those skilled in the art can understand meanings of the foregoing terms in the present disclosure according to specific conditions.

An electrocardiogram monitoring device 100 is provided according to embodiments of the present disclosure.

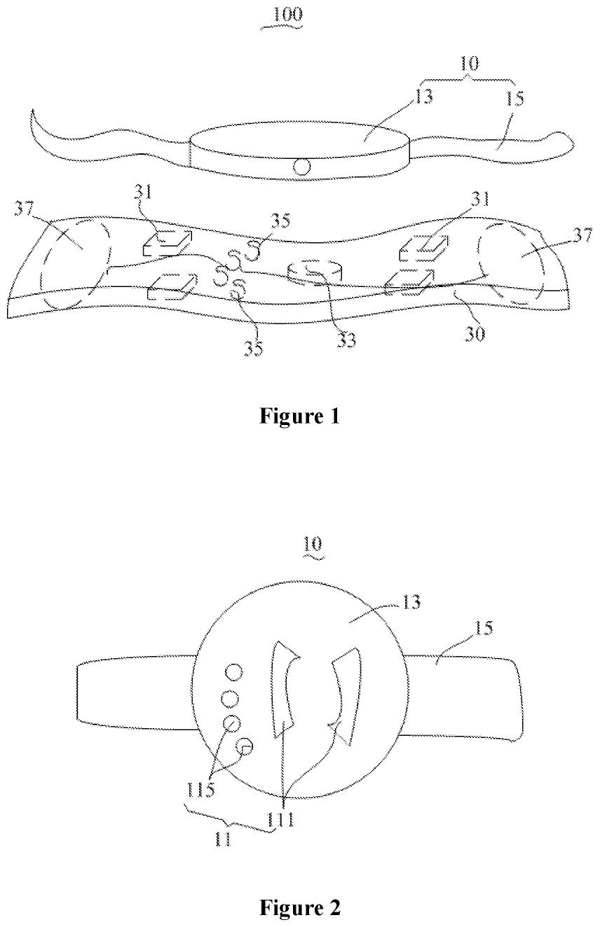

Reference is made to FIGS. 1 to 4. In an embodiment of the present disclosure, an electrocardiogram monitoring device 100 includes a main body 10 and a bottom support.

The main body 10 is configured to be worn on the wrist. The main body is provided with a first monitor 11. The first monitor is 11 configured to perform discontinuous electrocardiogram monitoring when being in contact with a finger.

The bottom support is connected with the main body 10, and is provided with a second monitor. The second monitor is configured to perform continuous electrocardiogram monitoring when being attached to a chest or a limb.

In the embodiment, the main body 10 may be a watch. The watch is provided with a movement and a pointer, and has a function of electrocardiogram recording. The watch may serve not only as a watch to present time, but also as an electrocardiogram monitoring tool for longtime wear, thus having various functions. The main body 10 is worn on the wrist, and hence does not interfere with normal life and work of a user. The electrocardiogram monitoring device 100 is capable to operate in various modes. First, the electrocardiogram monitoring device 100 may operate in a watch mode. That is, the main body 10 is worn on the wrist as an independent gadget, and implements discontinuous electrocardiogram monitoring, where “discontinuous” refers to temporal discontinuity. That is, the monitoring is performed only when the first monitor 11 of the main body 10 is in contact with a finger, and not performed when the first monitor 11 is not in contact with the finger, hence discontinuous in time. It is appreciated that such electrocardiogram monitoring may be continuous when performed during rest. In such case, the ECG (electrocardiogram) signal monitoring is performed inside the main body 10. A quantity of the bottom support(s) may be one or more, so that multiple monitoring modes may be implemented through coordination between the bottom support(s) with the main body 10, further improving functional diversity. In such case, the second monitor is coupled to a controller in signals, and the connection may be implemented electrical connection between the bottom support and the main body 10. When the second monitor of the bottom support is attached to the chest or the limb for a long time, the electrocardiogram monitoring device 100 operates in a “chest paste” mode or an electrode mode, and continuous electrocardiogram monitoring is performed for a time period. After receiving an electrocardiogram signal transmitted by the first monitor, the controller should perform a variety of processing on the signal. For example, the controller performs amplification, filtering, calibration, stabilization, and the like on the electrocardiogram signal, and then performs analog conversion to obtain electrocardiogram data suitable for analysis. It is appreciated that the case body 13 may be provided with a control button, and the first monitor 11 and the second monitor are not connected to each other unless the control button is pressed. In such case, the electrocardiogram signal is acquired only when a finger touches and keeps in contact with the first monitor, avoiding unnecessary monitoring data due to accidental touches. In addition, the case body 13 is further provided with a storage unit that is configured to receive and store the electrocardiogram data processed by the controller, so that the data may be subsequently provided to a user for check.

The electrocardiogram monitoring device 100 according to technical solutions of the present disclosure includes the main body 10 and the bottom support, and is capable of different monitoring modes. The main body 10 includes the controller and the first monitor 11. In daytime usage, the electrocardiogram monitoring device 100 may operate in a watch mode. The main body 10 may be worn on the wrist corresponding to a hand as an independent gadget, while a finger of the other hand touches the first monitor 11 of the main body 10 to perform electrocardiogram monitoring. The electrocardiogram signal generated by the first monitor 11 is transmitted to the controller for processing. The monitoring is stopped when inconvenient. The main body is worn on the wrist, thereby causing little inconvenience, improving user experience, and facilitating simple discontinuous electrocardiogram monitoring. In night usage or daytime rests, continuous electrocardiogram monitoring may be implemented through coordination of both hands without additional user operations. Alternatively, the main body 10 may be electrically connected to the bottom support, so that the second monitor on the bottom support is coupled to the controller in signals. After the second monitor is attached to the chest or the limb, an electrocardiogram signal is generated and transmitted to the controller to implement continuous electrocardiogram monitoring, which is called a “chest paste” mode or an electrode mode, without operation of two hands. Hence, the electrocardiogram monitoring device 100 can implement both continuous and discontinuous electrocardiogram monitoring, and is advantageous in flexibility, various selections, simplicity, and convenience.

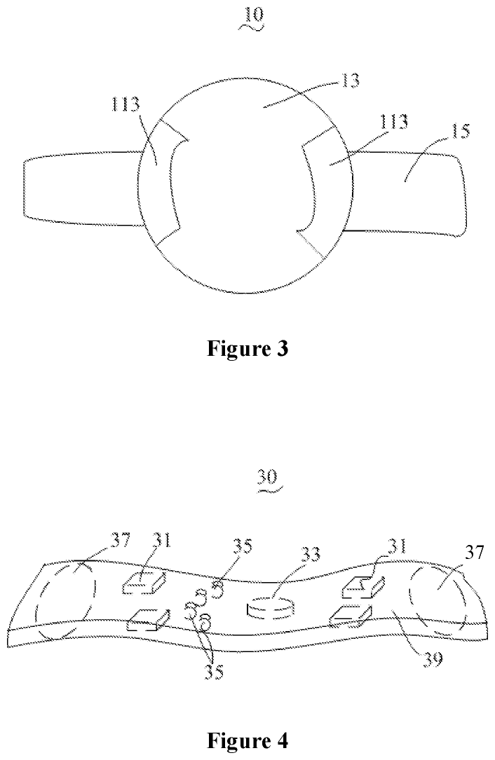

Reference is made to FIG. 2 in conjunction with FIG. 3. In an optional embodiment, the main body 10 includes the case body 13. The first monitor 11 includes an inside electrode 111, an outside electrode 113, and a connection contact 115. The inside electrode 111 is disposed on a surface, of the case body 13, attached to the wrist. The outside electrode 113 is disposed on a surface, of the case body 13, away from the wrist. The connection contact 115 is embedded inside the case body 13. The outside electrode 113 is connected to the inside electrode 111 via the connection contact 115.

Generally, a lead requires at least two electrodes. The electrodes are ECG (electrocardiogram) electrodes, which are specific electrocardiogram monitoring electrodes in medicine. In this embodiment, a shape of the case body 13 may be the same as that of a commercial watch case at present. For example, a cross section of the case body 13 may be square, circular, polygonal, or the like. Herein it is taken as an example that the cross section of the case body 13 is a circular. It can be understood that the main body 10 further includes a watch strap 15 connected between two ends of the case body 13, in order to form a band surrounding the wrist. When the main body 10 is worn on the wrist, for example, the left wrist, the electrocardiogram monitoring device 100 operates in the watch mode. The first monitor 11 of the main body 10 is provided with the inside electrode 111, the outside electrode 113, and the connection contact 115 connecting the inside electrode and the outside electrode. The inside electrode 111 is disposed on a surface, of the case body 13, attached to the wrist. Two inside electrodes 111 are arc-shaped and separated by a gap, in the middle of an inside surface of the case body 13, facilitating the case body sense pulses at the wrist and thereby improving accuracy of monitoring. The outside electrode 113 is disposed on the surface, of the case body 13, away from the wrist. Two outside electrodes 113 are disposed at a periphery of the case body 13. The outside electrodes 113 are connected to the inside electrodes 111 via the connection contact 115 embedded in the case body 13. Electrocardiogram monitoring on ECG signals is triggered when the right hand touches the outside electrode 113.

In an embodiment, the case body is configured to magnetically connect the bottom support.

In this embodiment, magnetic connection is provided between the case body 13 and the bottom support, which effectively improves efficiency of connecting the case body 13 and the bottom support. When the surface of the case body 13 approaches the bottom support, the case body 13 and the bottom support can be attracted to each other to achieve the connection, which is simple and convenient. One of the case body and the bottom support may be magnetic, while and the other may be ferritic, such that the connection is achieved through the magnetic attracting the ferritic. It is appreciated that both the case body and the bottom support may be magnetic as an alternative, and hence the case body and the bottom support attract each other.

Reference is made to FIG. 4. In an optional embodiment, the case body 13 is provided with at least one first magnet, and the bottom support is provided with a second magnet 31 corresponding to the first magnet.

In this embodiment, the case body 13 is provided with the first magnet, and the bottom support is provided with the second magnet 31, such that the connection between the two is improved through attraction between the two magnets. In addition, both a quantity of the first magnets and a quantity of the corresponding multiple second magnets 31 may be more than one. The multiple first magnets are uniformly arranged on the case body 13, and the multiple second magnets 31 are uniformly arranged on the bottom support, so as to achieve a uniform connection between the case body 13 and the bottom support. Structural stability is further improved.

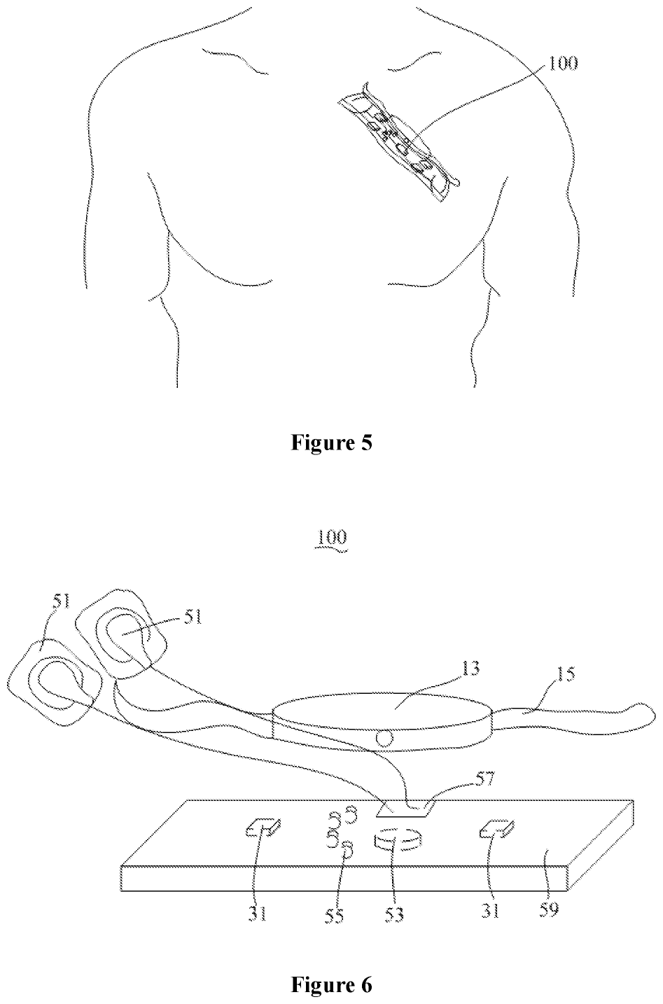

Reference is made to FIG. 1 in conjunction with FIG. 5. In an embodiment, the bottom support is a flexible bottom support 30. The main body 10 is configured to be attached to a surface of the flexible bottom support 30. The second monitor is disposed on a surface of the flexible bottom support 30 away from the main body 10.

In this embodiment, there may be two types of the bottom support, one of which is the flexible bottom support 30. The flexible bottom support 30 includes a flexible substrate 39. The flexible substrate 39 may be made of a soft material such as silica gel or rubber, to facilitate attachment between for the bottom support and the body, improving comfort of the user. The flexible bottom support 30 is in direct contact with the chest, and is applied to the “chest paste” mode. That is, when the user sleeps at night or the user lies flat in daytime, the second monitor is attached to the chest after the main body 10 is connected to the flexible bottom support 30. In this way, continuous electrocardiogram monitoring can be achieved without risks such as falling, and hence is convenient for the user. The flexible substrate 39 may be attached to the chest via a conductive gel. Hence, the attachment is firm, and electrical conduction is achieved between the second monitor and the body.

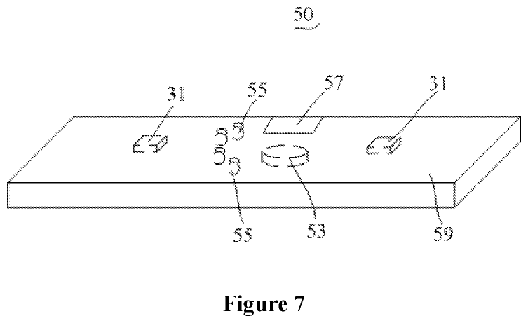

Reference is made to FIGS. 6 and 7. In an embodiment, the bottom support may alternatively be a rigid bottom support 50. The second monitor includes an electrode 51 for a lead. The electrode 51 is electrically connected to the rigid bottom support 50, and is configured to be attached to the chest or the limb.

In this embodiment, the main body 10 may be connected with the rigid bottom support 50, in order to realize accurate electrocardiogram monitoring. The rigid bottom support 50 includes a rigid substrate 59. The rigid substrate 59 may be made of a hard plastic material or metal materials. The rigid substrate 59 is not necessary to be in contact with the body, and may be placed in a pocket, placed on a table, or worn at the waist. The rigid bottom support 50 is connected to the electrode 51 for the lead. The electrode 51 may be attached to a left side and a right side of the chest, or connected to the limb. For example, the electrode 51 is attached to the left arm, the right arm, and the right leg. In this case, the electrocardiogram monitoring device 100 operates in the electrode mode, and can perform single-lead electrocardiogram monitoring. The single-lead electrocardiogram may provide necessary diagnostic data. The electrocardiogram monitoring device 100 has a simple structure and can be conveniently operated, and therefore is suitable for ordinary home usage. Such monitoring is close to professional electrocardiogram monitoring in hospital, and can obtain comprehensive monitoring data.

Reference is made to Figure in conjunction with FIG. 7. In an embodiment, the case body 13 further includes a sensor. The flexible bottom support 30 is provided with a first induction magnet 33 configured to magnetically attract the case body 13. The rigid bottom support 50 is provided with a second induction magnet 53 configured to magnetically attract the case body 13. The sensor is configured to detect whether magnetic attraction around the case body is between the case body and the first induction magnet 33, or between the case body and the second induction magnet 53.

In this embodiment, the case body 13 is provided with the sensor. The sensor is a Hall sensor. The flexible bottom support 30 is provided with the first induction magnet 33 matching with the Hall sensor. When the bottom support is attached to the case body 13 through attraction, the Hall sensor can detect whether the magnetic attraction is between the first magnet and the first induction magnet 33, or between the first magnet and the second induction magnet 53, so as to determine whether the connection is established between the main body 10 and the flexible bottom support 30, or between the main body 10 and the rigid bottom support 50. Hence, it can be determined whether the electrocardiogram monitoring device operates in the “chest paste” mode or the electrode mode, and vocal broadcast may be applied to prompt the user of a result of the determination, which improves user experience. The first induction magnet 33 may be configured to present one of the N-pole and the S-pole, of an induction magnet. The second induction magnet 53 may be configured to present the other of the N-pole and the S-pole, of an induction magnet.

In an optional embodiment, the second monitor includes two fixed electrodes 37. The two fixed electrodes 37 are disposed at two ends, respectively, of the flexible bottom support 30. The flexible bottom support 30 is further provided with a flexible expansion contact 35 electrically connected to the fixed electrodes 37. The flexible expansion contact 35 is disposed in correspondence to the connection contact 115, and is configured to connect the connection contact 115 electrically and elastically.

In this embodiment, the flexible bottom support 30 is provided with the flexible expansion contact 35, in order to implement electrical connection between the bottom support and the case body 13 when the electrocardiogram monitoring device 100 operates in the “chest patch” mode. The flexible expansion contact 35 is of a spring-loaded type, and matches with the connection contact 115 embedded in the case body 13. Thereby, the flexible expansion contact 35 press elastically against the connection contact 115, ensuring stability of electrical conduction. Both a quantity of the connection contacts 115 and a quantity of the corresponding flexible expansion contacts 35 may be more than one, so as improve the electrical conduction between the flexible bottom support 30 and the case body 13. The two fixed electrodes 37 are disposed at two ends, respectively, of the flexible bottom support 30, so as to increase an electrocardiogram voltage that is to be monitored, improving accuracy of the monitoring. It can be understood that a battery or a charging unit is provided within the case body 13. The battery or the charging unit may store sufficient power, and may supply the power to the bottom support after the bottom support is electrically connected to the case body 13. Thereby, it is ensured that the monitoring can be triggered when the fixed electrode 37 of the bottom support is in contact with the chest.

In an embodiment, the rigid bottom support 50 is provided with a rigid expansion contact 55 and a USB interface 57 that are electrically connected with each other. The rigid expansion contact 55 is disposed in correspondence to the connection contact 115, and is configured to connect the connection contact 115 electrically and elastically. The electrode 51 for the lead is electrically connected to the USB interface 57 via a connection wire.

In this embodiment, in a case that the electrocardiogram monitoring device 100 is required to operate in the electrode mode, the rigid bottom support 50 and the case body 13 are connected as described above. The rigid expansion contact 55 is configured to be a spring-loaded type, and is electrically connected to the connection contact 115, thus ensuring stability of the connection. The USB interface 57 is provided to electrically connect the rigid substrate 59 to the lead electrode 51, which facilitates connecting and disconnecting the lead electrode 51 to the rigid substrate 59, and arrangement and storage of the electrode 51 for the lead. The USB interface 57 may be of the Type-A, the Type-B, the Type-C, the Lightning, or the like, which is not limited herein.

In addition, the case body 13 may further be provided with a vibration motor, which may be configured to feed back a result of an operation. For example, when a control switch of the main body 10 is switched on, the vibration motor vibrates for 0.5 s, indicating that the monitoring system is powered. It is appreciated that the case body 13 may be provided with a display panel and a physical button, so that a monitored status can be displayed in real time and an abnormal situation can be adjusted or recorded based on a specific rule, which facilitates usage of the user.

An electrocardiogram monitoring system (not shown in the drawings) is further provided according to embodiments of the present disclosure. The electrocardiogram monitoring system includes an electrocardiogram monitoring device 100 and a display terminal coupled to the electrocardiogram monitoring device 100. Herein the electrocardiogram monitoring device 100 has been described as above. Since the electrocardiogram monitoring device 100 in the electrocardiogram monitoring system adopts all technical solutions of the foregoing embodiments, the electrocardiogram monitoring system according to embodiments of the present disclosure has all beneficial effects of the technical solutions of the foregoing embodiments, which are not repeated herein.

The display terminal in this embodiment may be a mobile terminal, such as a mobile phone, an Ipad, a laptop, a desktop, a liquid crystal display, or the like, which is not limited herein. As a preferable embodiment, the electrocardiogram monitoring device 100 is coupled to a mobile phone, so that a stored result of the monitoring may be transmitted to the mobile phone for view and analysis. The electrocardiogram monitoring device 100 may be connected to the mobile phone via a data cable. Alternatively, as a more convenience approach, the electrocardiogram monitoring device 100 may be provided with a Bluetooth™ module or a WiFi module to implement wireless communication between the electrocardiogram monitoring device 100 and the mobile phone.

The embodiments of the present disclosure are described in a progressive manner, and each embodiment places emphasis on the difference from other embodiments. Therefore, one embodiment can refer to other embodiments for the same or similar parts. Since the system disclosed in the embodiments corresponds to the devices disclosed in the embodiments, the description of the system is simple, and reference may be made to the relevant part of the devices.

As further be appreciated by those skilled in the art, the units and algorithmic steps in the examples described according to the embodiments disclosed herein can be implemented in forms of electronic hardware, computer software or the combination of the both. To illustrate the interchangeability of the hardware and the software clearly, the components and the steps in the examples are described generally according to functions in the above description. Whether hardware or software is used to implement the functions depends on a specific application and design constraints for the technical solution. For each specific application, different methods may be used by those skilled in the art to implement the described function, and such implementation should not be considered to depart from the scope of this invention.

The steps of the method or algorithm described according to the embodiments disclosed herein can be implemented in forms of hardware, a software module executed by a processor or the combination of the both. The software module may be stored in a Random Access Memory (RAM), a memory, a Read-Only Memory (ROM), an electrically programmable ROM, an electrically erasable programmable ROM, a register, a hardware disk, a movable magnetic disk, CD-ROM or any other forms of storage medium well known in the art.

It should be noted that, the relationship terms such as “first”, “second” and the like are only used herein to distinguish one entity or operation from another, rather than to necessitate or imply that an actual relationship or order exists between the entities or operations. Furthermore, the terms such as “include”, “comprise” or any other variants thereof means to be non-exclusive. Therefore, a process, a method, an article or a device including a series of elements include not only the disclosed elements but also other elements that are not clearly enumerated, or further include inherent elements of the process, the method, the article or the device. Unless expressively limited, the statement “including a . . . ” does not exclude the case that other similar elements may exist in the process, the method, the article or the device other than enumerated elements.

1. An electrocardiogram monitoring device, comprising:
a main body, configured to be worn on a wrist, wherein the main body is provided with a controller and a first monitor electrically coupled to the controller, the first monitor is configured to generate a first electrocardiogram signal and transmit the first electrocardiogram signal to the controller when being in contact with two hands; and
a bottom support, provided with a second monitor, wherein the second monitor is coupled to the controller in signals, and is configured to generate a second electrocardiogram signal and transmit the second electrocardiogram signal to the controller when being attached to a chest or a limb.

2. The electrocardiogram monitoring device according to claim 1, wherein:
the main body comprises a case body;
the first monitor comprises an inside electrode, an outside electrode, and a connection contact;
the inside electrode is disposed on a surface, of the case body, attached to the wrist;
the outside electrode is disposed on a surface, of the case body, away from the wrist;
the connection contact is embedded inside the case body; and
the outside electrode is electrically connected to the inside electrode via the connection contact.

3. The electrocardiogram monitoring device according to claim 2, wherein the case body is configured to magnetically connect to the bottom support.

4. The electrocardiogram monitoring device according to claim 3, wherein the case body is provided with at least one first magnet, and the bottom support is provided with a second magnet corresponding to the at least one first magnet.

5. The electrocardiogram monitoring device according to claim 2, wherein:
the bottom support comprises a flexible bottom support;
the main body is configured to be attached to a surface of the flexible bottom support; and
the second monitor is disposed on a surface of the flexible bottom support away from the main body when the main body is attached to the bottom support during operation of the electrocardiogram monitoring device.

6. The electrocardiogram monitoring device according to claim 5, wherein:
the bottom support further comprises a rigid bottom support;
the second monitor comprises an electrode for a lead; and
the electrode for the lead is electrically connected to the rigid bottom support and is configured to be attached to the chest or the limb.

7. The electrocardiogram monitoring device according to claim 6, wherein:
the case body further comprises a sensor;
the flexible bottom support is provided with a first induction magnet configured to magnetically attract the case body;
the rigid bottom support is provided with a second induction magnet configured to magnetically attract the case body; and
the sensor is configured to detect whether magnetic attraction around the case body is between the case body and the first induction magnet, or between the case body and the second induction magnet.

8. The electrocardiogram monitoring device according to claim 5, wherein:
the second monitor comprises two fixed electrodes, and the two fixed electrodes are disposed at two ends, respectively, of the flexible bottom support;
the flexible bottom support is further provided with a flexible expansion contact electrically connected to the fixed electrodes; and
the flexible expansion contact is disposed in correspondence to the connection contact, and is configured to connect the connection contact electrically and elastically.

9. The electrocardiogram monitoring device according to claim 6, wherein:
the rigid bottom support is provided with a rigid expansion contact and a USB interface that are electrically connected with each other;
the rigid expansion contact is disposed in correspondence to the connection contact, and is configured to connect the connection contact electrically and elastically; and
the electrode for the lead is electrically connected to the USB interface via a connection wire.

10. An electrocardiogram monitoring system, comprising:
the electrocardiogram monitoring device according to claim 1; and
a display terminal connected coupled to the electrocardiogram monitoring device.

11. The electrocardiogram monitoring device according claim 3, wherein:
the bottom support comprises a flexible bottom support;
the main body is configured to be attached to a surface of the flexible bottom support; and
the second monitor is disposed on a surface of the flexible bottom support away from the main body when the main body is attached to the bottom support during operation of the electrocardiogram monitoring device.

12. The electrocardiogram monitoring device according claim 4, wherein:
the bottom support comprises a flexible bottom support;
the main body is configured to be attached to a surface of the flexible bottom support; and
the second monitor is disposed on a surface of the flexible bottom support away from the main body when the main body is attached to the bottom support during operation of the electrocardiogram monitoring device.
